(19) United States
(12) Patent Application Publication
(10) Pub. No.: US 2026/0193615 A1
(43) Pub. Date: Jul. 9, 2026

(54) METHODS FOR CULTURING CELLS AND USES THEREOF

(71) Applicant: Acrocyte Therapeutics, Inc., New Taipei City (TW)

(72) Inventors: Ying-Chih Chang, Atherton, CA (US); Jia-Yang Chen, New Taipei City (TW); Yen-Jang Huang, New Taipei City (TW)

(21) Appl. No.: 19/441,284

(22) Filed: Jan. 6, 2026

Related U.S. Application Data
(60) Provisional application No. 63/742,121, filed on Jan. 6, 2025.

(51) Int. Cl. C12N 5/09

(52) U.S. Cl. CPC C12N 5/0694; C12N 2501/11; C12N 2501/115; C12N 2501/119; C12N 2501/155; C12N 2501/415; C12N 2513/00

(57) ABSTRACT

Provided is a method for culturing a sample of cells, which includes circulating epithelial cells, peripheral blood mononuclear cells (PBMC), or body fluid-derived cell samples (e.g. blood, ascites, pleural effusions, cerebrospinal fluids, perfusion fluids, sputum, and urine), or liquid biopsy-derived cell samples in presence of Bone Morphogentic Protein (BMP) inhibitor, a mitogenic growth factor, and a Wnt agonist, when culturing the circulating epithelial cells and relevant blood cells. Also provided is use of cell medium having a BMP inhibitor, a mitogenic growth factor, and a Wnt agonist in obtaining a cell culture of circulating epithelial cells, PBMC and body fluid-derived cells.

### CROSS-REFERENCE TO RELATED APPLICATION

This application claims the benefit of U.S. Provisional Application No. 63/742,121, filed on Jan. 6, 2025 under 35 USC § 119(e)(1), the subject matter of which is incorporated herein by reference.

### FIELD OF THE INVENTION

The present disclosure is related to an ex-vivo/in vitro culture method for circulating epithelial cells and blood cells. In particular, the present disclosure relates to maintaining and expanding epithelial cells and blood cells in ex vivo/in vitro culture condition.

### BACKGROUND OF THE INVENTION

Circulating tumor cells (CTCs) are tumor cells that extravasate from the primary tumor site and circulate in the bloodstream. At present, CTC has been used in many aspects of clinical research such as clinical diagnosis, risk assessment and decision-making, and treatment monitoring. However, CTC numbers ranging from 0 to 4 CTCs in 7.5 ml of blood indicate a good prognosis, while a cutoff of 5 CTCs in 7.5 ml of whole blood indicates a poor prognosis for breast cancer and prostate cancer. Since the CTC profiles are closer to the metastatic tumors than to the primary tumors, a more robust understanding of CTC biology is necessary for therapeutic approaches. But, CTCs are extremely rare in the peripheral bloodstream and are highly difficult to cultivate after enrichment. These increase the difficulty of CTC analysis after direct CTC enrichment.

Therefore, there is an urgent need for foundational advances to enrich and expand the rare population of cells including CTCs, and it will greatly improve the clinical application using these viable cells.

### BRIEF DESCRIPTION OF DRAWINGS

An understanding of the features and advantages of the present disclosure would be obtained by reference to the following detailed description that sets forth illustrative embodiments, in which the principles of the present disclosure may be utilized, and accompanying drawings of which:

FIG. 1 shows the bright field images of CTCs that cultured in accordance with the present disclosure for one week.

The FIGURE(S) herein are for illustrative purposes only and are not necessarily drawn to scale.

### DETAILED DESCRIPTION OF THE INVENTION

The present disclosure provides an effective strategy for providing functional autologous organoids (e.g., mesodermal organoids) via an organoid composite designed to promote the formation of organoids and spheroids from a single cell and adapted to use in autologous organoid therapies. Moreover, such autologous organoid therapies minimize the need for immunosuppression when treating diseased or injured organs during the rejuvenation process, offering an improved patient experience.

In one aspect, the present disclosure provides an in vitro method for obtaining cultured circulating epithelial cells, the method comprising:
providing a sample of cells, and
culturing the sample in a cell medium for a length of time sufficient to be able to observe formation of one or more cell spheroids having a diameter greater than 20 μm and optionally adherent cells and small cell spheroids having a diameter less than 20 μm, wherein the cell medium comprises:
   a Bone Morphogenetic Protein (BMP) Inhibitor, and
   a mitogenic growth factor, and
   a Wnt agonist, and
obtaining, from the cultured cell medium, the one or more cell spheroids containing the cultured circulating epithelial cells.

In one aspect, the present disclosure provides an in vitro method for obtaining cultured circulating epithelial cells, the method comprising:
providing the sample of cells, and
culturing the sample of cells in a cell medium for a length of time sufficient to be able to observe formation of adherent cells, small cell spheroids having a diameter less than 20 μm, and/or one or more cell spheroids having a diameter greater than 20 μm, wherein the cell medium comprises:
   a Bone Morphogenetic Protein (BMP) Inhibitor, and
   a mitogenic growth factor between 0.1 and 500 ng/ml, and
   a Wnt agonist, and
obtaining, from the cultured cell medium, the one or more cell spheroids containing the cultured circulating epithelial cells.

In one aspect, the present disclosure provides an in vitro method for obtaining cultured circulating epithelial cells, the method comprising:
providing a sample of cells, and
culturing the sample of cells in a cell medium for a length of time sufficient to be able to observe formation of adherent cells, small cell spheroids having a diameter less than 20 μm, and/or one or more cell spheroids having a diameter greater than 20 μm, wherein the cell medium comprises:
   a Bone Morphogenetic Protein (BMP) Inhibitor, and
   a mitogenic growth factor EGF between 0.1 and 500 ng/ml, and
   any one or a combination of Wnt agonist includes Wnt3, Wnt5, R-spondin 1, R-spondin 2, R-spondin 3 and R-spondin 4, Norrin, GSK inhibitor between 0.1 and 3000 ng/ml, and
   a B27 supplement and/or N2 supplement
obtaining, from the cultured cell medium, the one or more cell spheroids containing the cultured circulating epithelial cells.

In one aspect, the present disclosure provides an in vitro method for obtaining cultured circulating epithelial cells, the method comprising:
providing a sample of cells, and
culturing the sample of cells in a cell medium for a length of time sufficient to be able to observe formation of adherent cells, small cell spheroids having a diameter less than 20 μm, and/or one or more cell spheroids having a diameter greater than 20 μm, wherein the cell medium comprises:
   a Bone Morphogenetic Protein (BMP) Inhibitor between 0.1 and 500 ng/ml, and
   a mitogenic growth factor EGF between 0.1 and 500 ng/ml, and
   any one or a combination of Wnt agonist includes Wnt3, Wnt5, RSPO1 to RSPO4 between 1 and 3000 ng/ml, and
   a B27 supplement and/or an N2 supplement
obtaining, from the cultured cell medium, the one or more cell spheroids containing the cultured epithelial cells.

In one aspect, the present disclosure provides an in vitro method for obtaining cultured circulating epithelial cells, the method comprising:

providing a sample of cells in contact with a modified culture surface for organoid culture being micro-patterned surface or polyelectrolyte multi-layer surface, and
culturing the sample of cells in a cell medium for a length of time sufficient to be able to observe formation of adherent cells, small cell spheroids having a diameter less than 20 μm, and/or one or more cell spheroids having a diameter greater than 20 μm, wherein the cell medium comprises:
   a Bone Morphogenetic Protein (BMP) Inhibitor, and
   a mitogenic growth factor, and
   a Wnt agonist, and
obtaining, from the cultured cell medium, the one or more cells spheroids containing the cultured circulating epithelial cells.

In one aspect, the present disclosure provides an in vitro method for obtaining cultured circulating epithelial cells, the method comprising:
providing a sample of cells on a suspension cultured container,
culturing the sample of cells in a cell medium for a length of time sufficient to be able to observe formation of adherent cells, small cell spheroids having a diameter less than 20 μm, and/or one or more cell spheroids having a diameter greater than 20 μm, wherein cell medium comprises:
   a Bone Morphogenetic Protein (BMP) Inhibitor, and
   a mitogenic growth factor, and
   a Wnt agonist, and
obtaining, from the cultured cell medium, the one or more cells spheroids containing the cultured circulating epithelial cells.

In some embodiments, each of said one or more cell spheroids has any number of cells greater than 1, but is preferably at least 1×10¹ cells, at least 1×10² cells, 1×10³, 1×10⁴, 1×10⁵, 1×10⁶, 1×10⁷, 1×10⁸, or 1×10⁹ cells.

According to the present disclosure, the sample of cells comprises circulating epithelial cells, blood cells, peripheral blood mononuclear cells (PBMCs), or body fluid-derived cells, which are obtained and derived from a limited amount of body fluid. The body fluid-derived cells are for example but not limited to: a plurality of cells obtained from blood, ascites, pleural effusions, cerebrospinal fluids, perfusion fluids, sputum, saliva, sweat, and urine and any other materials comprising viable cells.

In some embodiments, the sample of cells is selected from the group consisting of whole blood sample, a buffy coat, peripheral blood mononuclear cells (PBMC) sample, a white blood cell sample, an apheresis product, and a leukapheresis product.

In some embodiments, the sample of cells is PBMCs, and the cell medium is supplied with a mitogenic growth factor, Wnt3a, R-spondin 3, and Noggin proteins under a culture condition in a 37° C., 5% CO² and about 20% oxygen cell culture incubator, which is normoxia. The cell medium is refreshed twice per week during the entire culture duration. After one week, CECs will be collected by gently pipetting for further bioassay.

In some embodiments, the sample of cells is obtained from 0.01 mL to 6 mL blood by removing red blood cells. In some embodiments, the method in accordance with the present disclosure comprises obtaining the sample of cells from blood at an amount about at least 0.01 mL, at least 0.5 mL or at least 0.1 mL; at an amount ranging from about 0.01 mL to 6 mL, 0.05 mL to 5 mL, 0.1 mL to 4 mL, 0.1 to 3 mL; or at an amount about 2 mL.

According to the present disclosure, the circulating tumor cells (CTC) that originate from carcinomas, wherein cancers of epithelial origin are the most prevalent as known as circulating epithelial cells and are anoikis-resistant and can survive in the bloodstream. A major population of CTCs substantially consists of CD45 negative, EpCAM positive and Cytokeratin (CK) positive cells. Remaining populations may be CK-negative CTCs and cytokeratin-positive and CD45-negative CTCs. In certain embodiments, the CTC is cytokeratin positive, EpCAM positive and CD45 negative cell having elevated nuclear-to-cytoplasmic (N:C) ratio of ≥0.5.

In some embodiments, the sample of cells is obtained from blood without depletion of normal blood cells (e.g., CD45⁺) to enrich for all remaining cells (including EpCAM-CTCs that have undergone EMT).

In some embodiments, the sample of cells may substantially consist of CTCs isolated from a blood sample. Several strategies are currently available for enrichment of CTCs. In certain embodiments, the mixture of primary cells is obtained by isolating viable CTCs from the rest of the blood constituents, such as platelets, red blood cells, and white blood cells, allowing CTCs concentration and thus facilitating the detection process. The enrichment step can be performed through three different types of technologies: protein expression-based, physical property-based and function-based technologies. Examples of strategies for viable circulating tumor cell (CTC) isolation include, but are not limited to, RosetteSep®, CTC-iChip, Ficoll®, Ficoll-Pacque®, Lymphoprep®, Percoll®, MetaCell®, Parsortix®, Collagen adhesion matrix assay (CAM) and Epithelial ImmunoSPOT assay (EPISPOT).

As used herein, the sample of cells to be cultured in the method of the present disclosure are derived and obtained from adult, i.e., the adult cells are adult epithelial or blood cells. In this context, “adult” means mature individual including newly-born baby or child, but adult cells exclude cells from embryonic or fetal organ or tissue. In a preferred embodiment, the adult cells are not derived from embryonic stem cells or embryonic stem cell lines, e.g., which have been differentiated in vitro, for example human embryonic stem cells or human embryonic stem cell lines.

According to the present disclosure, the small cell spheroids have a diameter less than 20 μm.

According to the present disclosure, each of the one or more cell spheroids has a diameter greater than 20 μm. In some embodiments, the one or more cell spheroids may be in a form of aggregation of cells and comprise cells having high nucleus-to-cytoplasm (N:C) ratio, which is an indicator of cell maturity, primarily because the nucleus typically decreases in size as a cell matures. For instance, the immature, “blast” forms of blood cells, such as erythrocytes, leukocytes, and megakaryocytes, begin with a high N:C ratio of 4:1. As these cells differentiate, this ratio decreases to 2:1 or even 1:1. Notable exceptions exist when mature lymphocytes often retain a high ratio (around 3:1 or even the original 4:1). Clinically, an increased N:C ratio is a common and important feature used to identify abnormal conditions, particularly precancerous dysplasia, and various forms of malignant cells.

According the present disclosure, the length of time is sufficient to be able to observe formation of adherent cells and one or more cell spheroids having a diameter greater than 20 μm in a cell medium. In some embodiments, the length of time is at least 3 days, at least 4 days, at least 5 days, at least 6 days, at least 7 days or at least 8 days. In one embodiment, the length of time is selected from the group consisting of: 48 hours to 1440 hours, 72 hours to 1080 hours, 84 hours to 720 hours, 96 hours to 600 hours, 108 hours to 480 hours, and 120 hours to 240 hours; 48 hours to 672 hours, 72 hours to 504 hours, 84 hours to 336 hours, 96 hours to 312 hours, 108 hours to 288 hours, 120 hours to 264 hours, and 144 hours to 240 hours. Alternatively, the length of time is selected from the group consisting of: 2 days to 14 days, 3 days to 10 days, 4 days to 9 days, and 5 days to 8 days. Alternatively, the length of time is no greater than 28 days, 21 days, 14 days, 13 days, 12 days, 11 days, 10 days, 9 days, 8 days, or 7 days.

In some embodiments, the step of culturing the sample of cells is performed in a cell medium for a length of time sufficient to observe formation of one or more cell spheroids having a diameter greater than 20 μm; and the length of time is selected from the group consisting of: 2 days to 14 days, 3 days to 10 days, 4 days to 9 days, and 5 days to 8 days.

In some embodiments, the BMP inhibitor is Noggin, the mitogenic growth factor is Epidermal Growth Factor (EGF).

In some embodiments, the BMP inhibitor comprises one or more of Noggin, Gremlin, Chordin, Follistatin, Twisted Gastrulation (Tsg), and Cerberus.

In some embodiments, the mitogenic growth factor comprises either one or a combination of growth factors selected from epidermal growth factor (EGF); platelet-derived growth factor (PDGF); insulin-like growth factor (IGF) family proteins including insulin, IGF1, and IGF2; fibroblast growth factor 1 sub-family including FGF1, FGF2; FGF3 sub-family including FGF3, FGF7, FGF10, and FGF22; FGF4 subfamily including FGF4, FGF5, and FGF6; transforming growth factor alpha (TGF-α); and transforming growth factor beta (TGF-β.

In some embodiments, the Wnt agonist comprises any one or a combination of R-spondin 1, R-spondin 2, R-spondin 3 and R-spondin 4, Wnt-3a, Wnt5a, Norrin, and GSK inhibitor.

In some embodiments, the cell medium further comprises a Rho-kinase (Rock) inhibitor selected from the group consisting of Y-27632, AT-13148, Fasudil, Ripasuldil, Netarsudil, H-1152.

In some embodiments, wherein the cell medium further comprises a Notch agonist consisting of Delta-like Ligand (DLL) 1, DLL3, DLL4, and Jagged Ligand 1 (Jagged1), Jagged Ligand 2 (Jagged2), dexamethasone, coelenterazine, tetracycline derivatives, and valproic acid.

In some embodiments, the cell medium further comprises one or more brain-derived neurotrophic factors selected from the group consisting of brain-derived neurotrophic factor (BDNF), nerve growth factor (NGF), and bovine pituitary extract (BPE).

In some embodiments, the cell medium further comprises either one or a combination of transferrin, selenium, ethanolamine, insulin, heparin, sonic-hedgehog, N-acetylcycteine, nicotinamide, neuregulin, cholera toxin, B27 supplement and/or N2 supplement.

In some embodiments, the BMP inhibitor comprises one or more of Noggin, Gremlin, Chordin, Follistatin, Twisted Gastrulation (Tsg), and Cerberus.

In some embodiments, the BMP inhibitor is at an amount ranging from 0.1 to 500 ng/ml.

According to the present disclosure, the step of culturing the sample of cells in a cell medium is for example but not limited to: culturing the sample of cells on a solid-liquid interface. In some embodiments, the solid-liquid interface includes a modified culture surface for organoid culture. The modified culture surface for organoid culture includes micro-patterned surface or polyelectrolyte complex surface. According to the present disclosure, the suspension cultured container refers to any method for culturing the sample of cells derived from body fluid in a suspension.

In some embodiments, the modified culture surface for organoid culture being micro-patterned surface or polyelectrolyte complex surface are modified with biomimetic materials, such as hydrogels, which fall broadly into three main species, each offering unique advantages for creating 3D cell culture environments. Natural hydrogels, derived from biological sources, inherently provide excellent biocompatibility and bioactivity due to their native components like cell-binding motifs and growth factors. Common examples include Basement Membrane Extracts (BME) and Matrigel®, rich in various ECM proteins; collagen, fibrin, any of glycoproteins and proteoglycans, which mimic structural components and wound healing matrices; and polysaccharides like alginate and hyaluronic acid, often modified to enhance cell adhesion. While highly biomimetic and supportive of complex cellular behaviors, their animal-derived origins can lead to batch-to-batch variability and undefined compositions.

In contrast, synthetic hydrogels are engineered polymers that offer precise control over their mechanical and biochemical properties, ensuring high reproducibility and defined compositions. Poly(ethylene glycol) (PEG) is a prime example, highly tunable in stiffness and biocompatibility, often functionalized with specific peptides to promote cell adhesion and enzymatic degradation. Other synthetic options like poly(vinyl alcohol) (PVA) and poly(acrylamide) (PAAm) also allow for tailored mechanical properties. The main limitation of purely synthetic hydrogels is their inherent bio-inertness, necessitating chemical modifications to support specific cell-material interactions.

Finally, hybrid hydrogels represent a rapidly advancing species, combining natural and synthetic components to leverage the best of both worlds. These semi-synthetic materials aim to achieve the desired bioactivity found in natural polymers with the tunable mechanical properties, reproducibility, and chemical definition of synthetic ones. By integrating elements like growth factors or cell-adhesion peptides (e.g., RGD sequences) into a robust synthetic scaffold (e.g., PEG-fibrinogen, GelMA-HA blends), hybrid hydrogels offer superior control over the cellular microenvironment, making them increasingly preferred for advanced applications in tumor organoid and stem cell research.

In some embodiments, the modified culture surface for organoid culture is a surface coated with polyelectrolyte multilayers (PEMs) and optionally an absorbent polymer. In some embodiments, the surface is coated with PEMs. In some embodiments, the surface is coated with PEMs and an absorbent polymer. PEMs disclosed herein comprise a plurality of alternating layers of oppositely charged polymers (1.e., polyelectrolytes). The oppositely charged polymers described herein comprise a combination of a positively charged polyelectrolyte (also referred to herein as a polycation) and a negatively charged polyelectrolyte (also referred to herein as a polyanion).

Exemplary polycations include, but are not limited to, poly(L-lysine) (PLL), poly(L-arginine) (PLA), poly(L-ornithine) (PLO), poly(L-histidine) (PLH), polyethyleneimine (PEI), poly[a-(4-aminobutyl)-L-glycolic acid] (PAGA), 2-(dimethylamino)ethyl methacrylate (DMAEMA), N,N-Diethylaminoethyl methacrylate (DEAEMA), and a combination thereof. In some instances, the polycation is PLL. In some instances, the polycation is PLO. In some instances, the polycation is PLH. In some instances, the polycation is PLA.

As used herein, ranges and amounts can be expressed as “about” a particular value or range. About also includes the exact amount. Hence “about 5 μL” means “about 5 μL” and also “5 μL.” The term “about” includes an amount that would be expected to be within experimental error.

As used herein, “substantially free,” as it relates to content of a substance in a composition, refers that the composition is generally in absence of the substance or comprises less than 1% of the substance by weight. In many embodiments, the composition contains less than 0.1%, less than 0.05%, less than 0.01% or less than 0.001% of the substance.

As used herein, the term “hypoxic” culture conditions as used herein refers to culture conditions in which cells are subjected to a reduction in available oxygen levels in the culture system relative to standard culture conditions in which cells are cultured at atmospheric oxygen levels (about 21 to allow 3D growth %). Non-hypoxic conditions are referred to herein as normal or normoxic culture conditions.

As used herein, the term “cell spheroid” or “spheroid” refers to an aggregate or assembly of cells cultured as opposed to growth as a monolayer. It is noted that the term “spheroid” does not imply that the aggregate is a geometric sphere. The aggregate may be highly organized with a well-defined morphology, or it may be an unorganized mass; it may include a single cell type or more than one cell type. The cells may be primary isolates, or a permanent cell line, or a combination of the two. Included in this definition are organoids and organotypic cultures.

As used herein, the term “organoid” as used herein refers to a heterogeneous 3D agglomeration of cells that recapitulates aspects of cellular self-organization, architecture and signaling interactions present in the native organ. The term “organoid” includes spheroids or cell clusters formed from suspension cell cultures.

The section headings used herein are for organizational purposes only and are not to be construed as limiting the subject matter described.

### EXAMPLES

### Example 1 Preparation of Tumor Spheroid Cultures

R3CE (Rapid, Reproducible, Rare Cell 3D Expansion) platform, a highly tunable thin films with adjustable properties, as a novel system for the expansion and maintenance of rare populated cells. R3CE is superior to the 2D culture in terms of easy operation, yields and closely mimicking patient conditions. It is clinically compatible with personalized drug testing and molecular analysis, providing timely guidance to the clinicians. Protein expression analysis revealed that CTCs cultured on our R3CE platform were able to maintain high levels of epithelial and marker properties, these were closer association to solid tumor in vivo.

The CTCs with or without enrichment from PBMC were incubated on R3CE 3D culture plate (Acrocyte, Taiwan) in DMEM/F12 culture medium supplied with 1% antibiotic-antimycotic (15240062, Gibco), 10% fetal bovine serum (35-010-CV, Corning), 50 ng/ml recombinant human epidermal growth factor protein (AF-100-15, Peprotech), 10 ng/ml recombinant human fibroblast growth factor-b (AF-100-18B, Peprotech), 10 ng/ml recombinant human fibroblast growth factor 10, and conditional medium with wnt3a, R-spondin 3, and noggin proteins under 3D culture condition in a 37° C., 5% CO₂ and about 20% oxygen cell culture incubator, which is normoxia. The complete culture medium will be refreshed twice per week during the entire culture duration. After one week, CTCs will be collected by gently pipetting the R3CE surface for further bioassay.

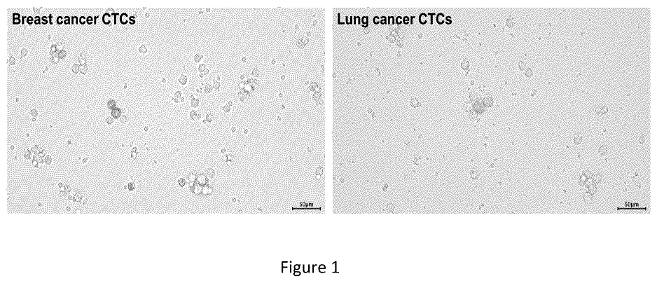

Thus, R3CE platform with the culture medium for ex vivo culture of CTCs from cancer patients was used. The CTCs from different cancer patients could form one or more 3D spheroids on our R3CE platform after 1 weeks (FIG. 1). As results in Table 1, the CTC numbers from PBMCs without ex-vivo culture were examined, and the initial CTC numbers were 0 to 28 CTCs in 2 mL blood. On contract, the cultured CTC numbers were expanded to 20-440 after 7 days culture in the 10 breast cancer patients. In other words, CTCs could proliferate and expand approximately 1 to several hundred folds by the method of the present disclosure in the culture medium on R3CE platform. Since other blood cells did not proliferate on R3CE platform, the purity of CTC was greatly improved, which increased the reliability of bio-analysis.

TABLE 1
The CTC numbers before/after subject to the method in accordance with the present disclosure for one week, and related
patient clinical information.

| | | B | C | | | | | | | | | | | |
|---|---|---|---|---|---|---|---|---|---|---|---|---|---|---|
| | A | Initial | Cultured | D (C/D) | | | | H | I | J | K | | | N |
| | Case | CTCs/ | CTCs/ | Proliferation | E | F | G | Pathology | Molecular | ER | PR | L | M | Ki67 |
| | number | 2 ml blood | 2 ml blood | rate | Sex | Age | Stage | Grade | type | (%) | (%) | HER2 | FISH | (%) |
| 1 | Patient 1 | 28 | 26 | 1 | Female | 53 | IIIB | II | TNBC | — | — | 1+ | | 70 |
| 2 | Patient 2 | 1 | 20 | 20 | Female | 59 | IIIB | II | HER2 | — | — | 3+ | | 22 |
| 3 | Patient 3 | 1 | 30 | 30 | Female | 35 | IA | II | B2 | 40 | 70 | 3+ | | 25 |
| 4 | Patient 4 | 0 | 104 | — | Female | 49 | IIB | III | HER2 | — | — | 2+ | + | 50 |
| 5 | Patient 5 | 1 | 440 | 440 | Female | 50 | IIIB | III | TNBC | — | — | 1+ | | 70 |
| 6 | Patient 6 | 4 | 100 | 25 | Female | 59 | IIB | III | HER2 | 2 | — | 3+ | | 70 |
| 7 | Patient 7 | 9 | 80 | 9 | Female | 68 | IB | III | B2 | 95 | 60 | 2+ | + | 50 |
| 8 | Patient 8 | 5 | 130 | 26 | Female | 43 | IB | II | HER2 | 90 | 5 | 3+ | | 40 |
| 9 | Patient 9 | 5 | 20 | 4 | Female | 58 | IIA | II | B2 | 95 | 10 | 2+ | + | 18 |
| 10 | Patient 10 | 8 | 40 | 5 | Female | 57 | IIA | II | TNBC | — | — | 0 | | 90 |

1. An in vitro method for obtaining cultured circulating epithelial cells, the method comprising:
providing a sample of cells including circulating epithelial cells, peripheral blood mononuclear cells (PBMCs), or body fluid-derived cells, and
culturing the sample of cells in a cell medium for a length of time sufficient to observe formation of one or more cell spheroids having a diameter greater than 20 μm, wherein the cell medium comprises:
a Bone Morphogenetic Protein (BMP) Inhibitor, and
a mitogenic growth factor, and
a Wnt agonist, and
obtaining, from the cultured cell medium, the one or more cell spheroids containing the cultured circulating epithelial cells.

2. The method according to claim 1, wherein the body fluid is selected from the group consisting of: blood, ascites, pleural effusion, cerebrospinal fluid, perfusion fluid, sputum, and urine.

3. The method according to claim 1, wherein the length of time is no greater than 10 days.

4. The method according to claim 1, which comprises providing the sample of cells using between 0.01 mL and 6 mL of blood.

5. The method according to claim 1, wherein the cell spheroids are selected from the group consisting of mammary spheroids, pulmonary spheroids, hepatic spheroids, kidney spheroids, cerebral spheroids, cerebellar spheroids, intestinal spheroids, gastric spheroids, pancreatic spheroids, prostate spheroids, cardiac spheroids, vascular spheroids, skeletal muscle spheroids, and retinal spheroids.

6. The method according to claim 1, wherein the Wnt agonist comprises any one or a combination of R-spondin 1, R-spondin 2, R-spondin 3 and R-spondin 4, Wnt-3a, Wnt5a, Norrin, and GSK inhibitor.

7. The method according to claim 1, wherein the Wnt agonist comprises either one or a combination of R-spondin 1, R-spondin 2, R-spondin 3 and R-spondin 4, Norrin, Wnt3a, Wnt5a, and GSK-inhibitor including LY2090314, CHIR99021, AZD1080, AR-A014418, Tideglusib, Elraglusib, L803-mts, BAY 1125976, or TWS119 at an amount between 0.1 and 3000 ng/ml.

8. The method according to claim 1, wherein the BMP inhibitor comprises either one or a combination of Noggin, Gremlin, Chordin, Follistatin, Twisted Gastrulation (Tsg), and Cerberus.

9. The method according to claim 7, wherein the BMP inhibitor is at an amount between 0.1 and 500 ng/ml.

10. The method according to claim 1, wherein the mitogenic growth factor comprises either one or a combination of growth factors selected from epidermal growth factor (EGF); platelet-derived growth factor (PDGF); insulin-like growth factor (IGF) family proteins including insulin, IGF1, and IGF2; fibroblast growth factor 1 sub-family including FGF1, FGF2; FGF3 sub-family including FGF3, FGF7, FGF10, and FGF22; FGF4 subfamily including FGF4, FGF5, and FGF6; transforming growth factor alpha (TGF-α); and transforming growth factor beta (TGF-β).

11. The method according to claim 9, wherein the mitogenic growth factor is at an amount between 0.1 and 500 ng/ml.

12. The method according to claim 1, wherein the cell medium further comprises a Rho-kinase (Rock) inhibitor selected from the group consisting of Y-27632, AT-13148, Fasudil, Ripasuldil, Netarsudil, H-1152.

13. The method according to claim 1, where in the cell medium further comprises a Notch agonist consisting of Delta-like Ligand (DLL) 1, DLL3, DLL4, and Jagged Ligand 1 (Jagged1), Jagged Ligand 2 (Jagged2), dexamethasone, coelenterazine, tetracycline derivatives, and valproic acid.

14. The method according to claim 1, where in the cell medium further comprises one or more brain-derived neurotrophic factors selected from the group consisting of brain-derived neurotrophic factor (BDNF), nerve growth factor (NGF), and bovine pituitary extract (BPE).

15. The method according to claim 1, where in the cell medium further comprises B27 supplement and/or N2 supplement.

16. An in vitro method for obtaining cultured circulating epithelial cells, the method comprising: providing a sample of cells including circulating epithelial cells, peripheral blood mononuclear cells (PBMCs), or body fluid-derived cells, and
culturing the sample of cells in a cell medium for a length of time sufficient to be able to observe formation of one or more cell spheroids having a diameter greater than 20 μm, wherein the cell medium comprises:
a Bone Morphogenetic Protein (BMP) Inhibitor, and
a mitogenic growth factor EGF between 0.1 and 500 ng/ml, and
any one or a combination of Wnt agonist includes Wnt3, Wnt5, R-spondin 1, R-spondin 2, R-spondin 3 and R-spondin 4, Norrin, GSK inhibitor between 0.1 and 3000 ng/ml, and
a B27 supplement and/or N2 supplement,
obtaining, from the cultured cell medium, the one or more cell spheroids containing the cultured circulating epithelial cells.

17. An in vitro method for obtaining cultured circulating epithelial cells, the method comprising:
providing a sample of cells including circulating epithelial cells, peripheral blood mononuclear cells (PBMCs), or body fluid-derived cells, and
culturing the sample of cells in a cell medium for a length of time sufficient to be able to observe formation of one or more cell spheroids having a diameter greater than 20 μm, wherein the cell medium comprises:
a Bone Morphogenetic Protein (BMP) Inhibitor between 0.1 and 500 ng/ml, and
a mitogenic growth factor including EGF between 0.1 and 500 ng/mL, and
any one or a combination of Wnt agonist includes Wnt3, Wnt5, RSPO1 to RSPO4 between 1 and 3000 ng/ml, and
a B27 supplement and/or an N2 supplement
obtaining, from the cultured cell medium, the one or more cell spheroids containing the cultured circulating epithelial cells.
